(19) United States
(12) Patent Application Publication
(10) Pub. No.: US 2025/0228468 A1
(43) Pub. Date: Jul. 17, 2025

(54) Simultaneous Magnetic Resonance Multislice Imaging Technique with Adapted Recording Sequence

(71) Applicant: Siemens Healthineers AG, Forchheim (DE)

(72) Inventors: Flavio Carinci, Würzburg (DE); Dominik Paul, Bubenreuth (DE); Mario Zeller, Erlangen (DE)

(73) Assignee: Siemens Healthineers AG, Forchheim (DE)

(21) Appl. No.: 19/027,446

(22) Filed: Jan. 17, 2025

(30) Foreign Application Priority Data
Jan. 17, 2024 (DE) 10 2024 200 392.1

(51) Int. Cl. A61B 5/055

(52) U.S. Cl. CPC A61B 5/055

(57) ABSTRACT

In a method for operating a MR facility for recording a MR dataset using a simultaneous multislice imaging technique, a recording sequence is established using an ordering rule. The recording sequence includes an allocation of slices to corresponding sequence portions of at least one repetition sequence covering all the slices of at least one allocated concatenation. A crosstalk condition for the established recording sequence is evaluated, which checks whether at least one first slice that is recorded in the last sequence portion of a repetition sequence is spatially adjacent to at least one second slice which is recorded in the first sequence portion of the same repetition sequence. The recording sequence may be adapted, based on the crosstalk condition, to increase the temporal separation between the recording of the first and second slices. The adapting may include displacing a slice group to a central sequence portion of the repetition sequence.

### CROSS REFERENCE TO RELATED APPLICATIONS

This patent application claims priority to German (DE) patent application Ser. No. 102024200392.1, filed Jan. 17, 2024, which is incorporated herein by reference in its entirety.

### BACKGROUND

### Field

The disclosure relates to a computer-implemented method for operating a magnetic resonance facility for recording a magnetic resonance dataset, a magnetic resonance facility, a computer program and an electronically readable data carrier.

### Related Art

Magnetic resonance imaging is an established imaging method, particularly in medical engineering. Efforts to improve the various imaging techniques are directed, above all, toward image quality, but also toward reducing the, sometimes very long, recording times. One of the key elements for optimizing the recording times is the parallel recording of a plurality of slices.

In order to accelerate magnetic resonance tomography, for example, the simultaneous multi-slice (SMS) method has been proposed. With this, a number of slices specified by an acceleration factor, for example, two or three slices are at least substantially simultaneously excited and simultaneously read out. SMS techniques are described, for example, in an article by Markus Barth et al., “Simultaneous Multi Slice (SMS) Imaging Techniques”, Magn Reson Med 75 (2016), pages 63-81. The at least substantially simultaneously excited slices are “collapsed” into a single image, that is, the magnetic resonance signal acquisition comprises information from all these slices. The magnetic resonance data of the simultaneously acquired slices can be separated during the postprocessing with the aid of separation algorithms, for example slice-GRAPPA (see the article by K. Setsompop et al., “Blipped-Controlled Aliasing in Parallel Imaging (blipped-CAIPI) for Simultaneous Multi-Slice EPI with Reduced g-Factor Penalty”, Magn Reson Med 67 (2012), pages 1210-1224).

A known problem in SMS imaging is so-called slice crosstalk. When a slice is excited, the longitudinal magnetization in the adjacent slices is also partially saturated. The reason for this effect lies in the fact that the slice excitation profile is not a perfect rectangular function and overlaps with the adjoining slices. In order to solve this problem, it has been proposed to select the temporal acquisition sequence for the slices in such a way that the slice excitations are interleaved, which means that adjacent slices are not excited immediately one after another, rather by way of the recording sequence (which can also be referred to as the “reordering scheme”), a time delay is brought about. If, for example, a total of thirteen slices are numbered according to their spatial arrangement, using a recording sequence “S1 S2 S3 S4 S5 S6 S7 S8 S9 S10 S11 S12 S13”, would lead to slice crosstalk. If, however, a temporally interleaved recording sequence such as “S1 S3 S5 S7 S9 S11 S13 S2 S4 S6 S8 S10 S12” were to be used, spatially adjacent slices such as S1 and S2 are never excited sequentially.

However, in SMS imaging, it is not always possible to prevent crosstalk between the slices by the new ordering of the slices, since slices that are at different locations in the space are excited simultaneously. For example, problems always occur if the total number of slices, the number of concatenations (concats for short) and the acceleration factor (SMS factor) fulfills the crosstalk condition that the number of concatenations is greater than one and a reduced slice count, which is given by the total number of the slices divided by the acceleration factor, modulo the concatenation number is equal to one.

This is explained using the example of 26 slices, two concatenations and an acceleration factor of two. As slice groups of slices to be excited simultaneously, (S1,S14), (S2,S15), (S3, S16), (S4,S17), . . . are formed, so that for the first concatenations in the interleaved reordering scheme, an allocation of slice groups to sequence portions results in a repetition sequence (S1,S14), (S3,S16), (S5,S18), (S7,S20), (S9,S22), (S11,S24), (S13,S26). This has one sequence portion more than the second concatenation with the repetition sequence (S2,S15), (S4,S17), (S6,S19), (S8,S21), (S10,S23), (S12,S25).

Since a plurality of repetitions of a repetition sequence are often used for recording, after completion of a repetition of the first concatenation with the slices S13 and S26, the next repetition would begin with the slices S1 and S14, so that the spatially adjacent slices S13 and S14 are excited immediately one after another in the first concatenation.

The slice S13 is excited in the last sequence portion of the first repetition of the repetition sequence and the slice S14 is excited in the first sequence portion of the second repetition of the repetition sequence. This leads to slice crosstalk, which results in an artifact in the final magnetic resonance dataset. In the example described, the slice S14 would appear darker than all the other slices since its magnetization has been partially saturated by the excitation of the slice S13 recorded immediately before it.

EP 4 104 755 A1 describes a method of this type, in order to solve this problem, it is proposed to exchange the last slice group or the first slice group of the affected concatenation with another slice group in the same or another concatenation, in particular with the respective immediately adjacent (i.e. penultimate or second) slice group of the same concatenation. With that an excellent reduction in the slice crosstalk for protons bound within water is achieved.

However, the problem is not solved if a fat saturation technique or inversion recovery is applied to protons bound within fat. Experiments have revealed, for example, by a comparison of the recordings with a water phantom and an oil phantom, that in the oil phantom, despite swapping the first two or the last two slice groups of the concatenation concerned, a marked darkening of a slice occurred due to slice crosstalk. This has its origin ultimately in the fact that an effective fat saturation is dependent upon the steady state of the fat signal/fat tissue. Both the slice crosstalk and also the slice reordering influence this steady state, for which reason the aforementioned effect can occur.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows excitation profiles of adjacent slices.

FIG. 2 shows a schematic illustration of a slice crosstalk artifact.

FIG. 3 shows a flow diagram of an exemplary embodiment of the method according to the disclosure.

FIG. 4 shows a re-iterated repetition sequence of a recording sequence before an adaptation.

FIG. 5 shows the re-iterated repetition sequence after the adaptation.

FIG. 6 shows a magnetic resonance facility according to the disclosure.

FIG. 7 shows the functional structure of a control facility of the magnetic resonance facility.

The exemplary embodiments of the present disclosure will be described with reference to the accompanying drawings. Elements, features and components that are identical, functionally identical and have the same effect are—insofar as is not stated otherwise-respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

It is therefore an object of the disclosure to provide an improved, easily implementable, effective possibility for reducing slice crosstalk that is also effective when applying fat saturation and inversion recovery modules to slices with protons bound into fat.

This object is achieved according to the disclosure with a computer-implemented method, a magnetic resonance apparatus, a computer program and an electronically readable data carrier according to one or more exemplary embodiments.

Aspects of the disclosure relate to a computer-implemented method for operating a magnetic resonance facility for recording a magnetic resonance dataset which comprises a total number of slices, using a simultaneous multislice imaging technique, wherein in a sequence portion of the magnetic resonance sequence that is used, magnetic resonance signals are simultaneously acquired from a simultaneity number, that is equal to an acceleration factor and is at least two, of slices of a slice group, wherein
a recording sequence comprising an allocation of slices to corresponding sequence portions of at least one repetition sequence which covers all the slices of at least one allocated concatenation, using at least one ordering rule is established, and
a crosstalk condition for the established recording sequence is evaluated, wherein the crosstalk condition checks whether at least one first slice that is recorded in the last sequence portion of at least one of the at least one repetition sequence is spatially adjacent, in particular spatially immediately adjacent, to at least one second slice which is recorded in the first sequence portion of the same repetition sequence, and
on fulfillment of the crosstalk condition, the recording sequence is adapted to increase the temporal separation between the recording of the first and the second slice.

In addition, the disclosure relates to a magnetic resonance facility, a computer program and an electronically readable data carrier.

In a method of the aforementioned type, it is provided according to the disclosure that for adapting the recording sequence, the slice group comprising the first slice or the slice group comprising the second slice is displaced to a central sequence portion, in particular into the exact center, of the repetition sequence.

It is proposed to displace one of the problematic, temporally adjacently recorded and spatially adjacent slices to a central position, and therefore a central sequence portion, of the repetition sequence of the affected concatenation (typically the concatenation with the most slice groups). The sequence of the recording of the other slices and/or slice groups is therein not changed. This is carried out for each repetition. Although fundamentally therein, a central sequence portion can be broadly defined, in particular, as at least two sequence portions removed from the first or the last sequence portion of the repetition sequence and/or exactly centrally, it is still particularly preferred to select the exact center, since then the greatest possible separation is placed between the recording of the first slice and the second slice. Since the concatenation with the greatest number of slice groups in which the problem usually occurs mostly covers an odd number of slice groups and thus contains an odd number of sequence portions, an exact center of the repetition sequence is clearly defined so that the slice group of the first slice or the slice group of the second slice can be displaced without difficulty and without changing the remaining sequence of the slice groups in the repetition sequence to this exactly central sequence portion. In cases of an even number of sequence portions of the concatenation concerned, the central sequence portion of the two candidates that is temporally further from the recording of each non-displaced slice is selected as the target of the displacement. An adaptation is only carried out in the concatenation concerned.

By this means, a clear reduction of cross-talk and thus of crosstalk artifacts, is achieved. This applies, in particular, for slices and/or recording regions containing mainly fat, specifically protons bound into fat, so that a suitable development of the present disclosure provides that in the magnetic resonance sequence, a preparation module with at least one preparation pulse is used as the excitation pulse and/or the second slice has a high fat content. Particularly when preparation techniques are used, in particular, on protons bound in fat, it has been found that crosstalking magnetization effects remain sufficiently long to cause crosstalk artifacts, in particular, a darkening of the second slice if the conventional techniques and thus, in particular, only an exchange with the nearest neighbor are applied in the temporal sequence. By way of the maximization of the temporal separation of the recording of the first and second slices, however, the disclosure enables a marked improvement in the image quality by reducing, in particular entirely preventing, crosstalk artifacts.

The preparation module can be, in particular, a fat saturation module and/or an inversion recovery module and/or a SPAIR (SPectral Attenuated Inversion Recovery) module. For these cases in which, using the conventional procedure, crosstalk artifacts have still arisen, with the procedure according to the disclosure, excellent results, in particular, entirely or at least almost artifact-free results, have been obtained.

The establishment of the recording sequence and the checking of the crosstalk condition can be configured in the context of the present disclosure as previously described in EP 4 104 755 A1.

Specifically, it can be provided that if the slices are numbered according to their spatial arrangement in at least one stacking direction and a concatenation number of concatenations is used, wherein the ordering rule defines a simultaneous recording of a plurality of slices in slice groups such that the slice numbers of slices in each slice group differ by the total number divided by the acceleration factor so that a list of slice groups results that is sorted in ascending or descending manner according to their lowest slice number, wherein, in order to distribute the slice groups to concatenations and sequence portions,
if the concatenation number is one, two sublists are created one after another, wherein one sublist contains all the even slice numbers and the other sublist contains all the odd slice numbers, and
if the concatenation number is greater than one, slice groups are allocated according to the list, one after another, to different concatenations in a particular concatenation sequence.

It should be noted herein that, when these ordering rules are applied, the crosstalk condition is not fulfilled in any case if, in each of the plurality of concatenations, the same number of slices is acquired. If, for example, a total number of 26 slices is to be acquired with an acceleration factor of two and a concatenation number of three, the repetition sequences for each concatenation would comprise the following slice groups in temporal sequence:
Concatenation K1: (S1,S14), (S4,S17), (S7,S20), (S10,S23), (S13,S26)
Concatenation K2: (S2,S15), (S5,S18), (S8,S21), (S11,S24)
Concatenation K3: (S3,S16), (S6,S19), (S9,S22), (S12,S25).

As can be seen, the last sequence portion in the repetition sequence for the concatenation K1 may comprise the slice S13, whereas the first sequence portion may comprise the slice S14. On re-iteration of the repetition, spatially immediately adjacent slices S13 and S14 would therefore be acquired (and thus excited) in a temporally immediately adjacent manner. A comparable slice crosstalk risk arises, for example
for a total number of 39 slices, an acceleration factor of three and a concatenation number of two,
for a total number of 38 slices, an acceleration factor of two and a concatenation number of two, and
for a total number of 39 slices, an acceleration factor of three and a concatenation number of three.

When using these ordering rules, it has been ascertained that slice crosstalk occurs if a simple mathematical relationship obtains the value “true”. In particular, in exemplary embodiments, a reduced number of slices is defined as the total number divided by the acceleration factor and, in order to evaluate the crosstalk condition, it is checked whether a first integer number defined as the reduced number of slices modulo a second integer number, defined as the concatenation number, is equal to one.

This means that in this often-used ordering scheme, the aforementioned slice crosstalk problem arises only with special combinations of the total number of slices (NTotalSlices), the acceleration factor (SMSfactor) and the concatenation number (NConcatenations). These combinations are defined through the fulfillment of the following logical mathematical relation:

NReducedSlices % NConcatenations==1,
where the symbol % represents a modulo operation, and NReducedSlices=NTotalSlices/SMSfactor. All the variables mentioned above are whole numbers greater than zero, wherein the number of concatenations is greater than one.

For the evaluation of the crosstalk condition, in this embodiment, it is checked whether the current combination of the total number of slices (NTotalSlices), the acceleration factor (SMSfactor) and the concatenation number (NConcatenations) would lead to a slice crosstalk in that it is checked whether the described logical mathematical relation NReducedSlices % NConcatenations==1 is true. If yes, the adaptation rule described above is applied, according to which a displacement of the slice group of the first slice or the second slice into the center of the repetition sequence takes place without changing the order of the other slice groups.

The disclosure further relates to a magnetic resonance facility comprising a control facility (controller) that may be configured to carry out the steps of a method according to the disclosure. All the embodiments and features relating to the method according to the disclosure can be transferred analogously to the magnetic resonance facility according to the disclosure, so that the aforementioned advantages can also be achieved therewith.

In particular, the control facility can comprise at least one processor and at least one memory storage means. Functional units for carrying out steps of the method according to the disclosure can be implemented by way of hardware and/or software. In particular, the control facility can comprise an ordering unit for applying the at least one ordering rule for determining the recording sequence, a condition unit for evaluating the crosstalk condition and an adapting unit for adapting the acquisition sequence by displacement to a central sequence portion of the repetition sequence if the crosstalk condition is fulfilled. For subsequent recording of the magnetic resonance dataset, the control facility can also comprise a sequence unit, as is known in principle.

A computer program according to the disclosure can be loaded directly into a storage means of a control facility of a magnetic resonance facility and has program means which, when the computer program is executed in the control facility, causes it to carry out the steps of the method according to the disclosure. The computer program can be stored on an electronically readable data carrier according to the disclosure which therefore has control information stored thereon, which comprises at least one computer program according to the disclosure and is configured such that, when the data carrier is used in a control facility of a magnetic resonance facility, said control facility is configured to carry out a method according to the disclosure. The data carrier can be, in particular, a non-transient data carrier, for example, a CD-ROM.

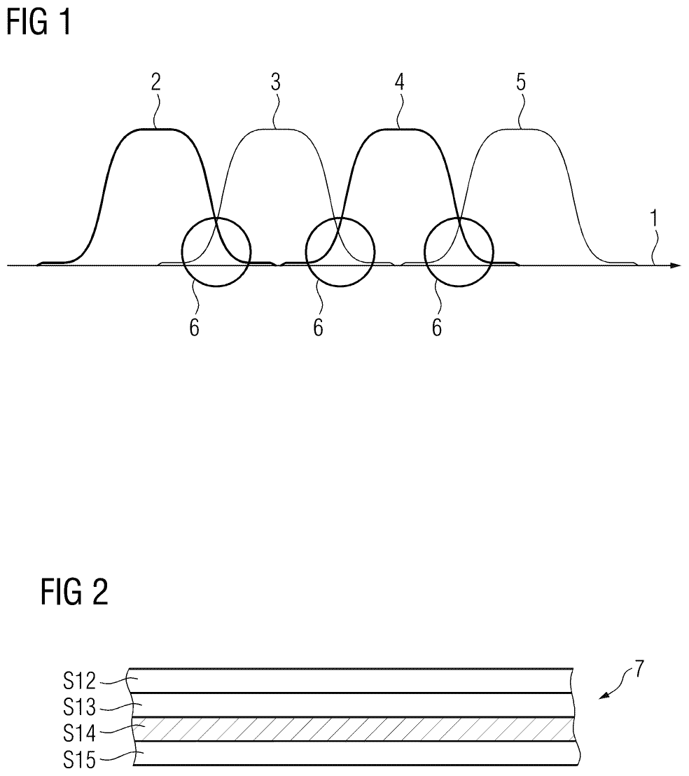

FIG. 1 shows schematically the problem of crosstalk between spatially adjacent slices in a stacking direction 1 due to excitation profiles 2, 3, 4 and 5. The excitation profiles 2 to 5 are not perfectly rectangular, so that they overlap in the region of adjacent slices, in crosstalk regions 6. If therefore magnetic resonance signals are recorded from a plurality of slices in a slice stack, the temporally adjacent excitation of spatially immediately adjacent slices is avoided, for example by way of interleaving, as described above.

The crosstalk problem can also occur in simultaneous multislice (SMS) imaging, and even if an ordering scheme, written by way of at least one ordering rule, is utilized to determine a recording sequence. This is always the case if in one of a plurality of concatenations in the associated repetition sequence, in the last sequence portion a first slice and, in the first sequence portion, a second slice are recorded which are spatially immediately adjacent, as set out in examples above. In a plurality of repetitions of the repetition sequence, crosstalk can then occur and can lead to a crosstalk artifact, as indicated, by way of example, in FIG. 2. FIG. 2 shows, for both the examples set out above of a slice stack 7 of twenty-six slices, the slices S12 to S15, wherein by way of the excitation of the slice S13 in the last sequence portion, slice crosstalk arises for slice S14 in the subsequent first sequence portion of the next repetition and this slice is significantly darker than all the other slices.

It has now been found that slice crosstalk and the associated crosstalk artifacts can still occur, in particular when preparation modules are used, such as, fat saturation modules and/or inversion recovery modules and/or SPAIR modules in combination with protons bound in fat, even on exchange of the last or first slice group with the penultimate and/or second slice group as an adaptation rule for adapting the recording sequence. In particular, in such cases, the crosstalk persists longer than the duration of a sequence portion.

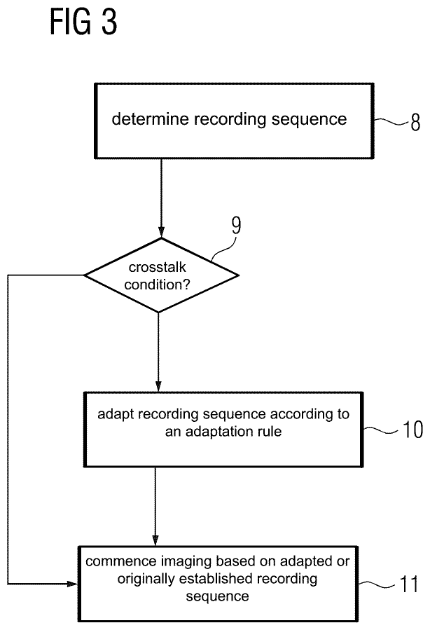

In such cases, in order also to be able to avoid, or at least reduce, crosstalk artifacts, a method such as that described in FIG. 3 is applied.

Therein, in a first step 8, the recording sequence is determined, as is conventionally known. In this case, ordering rules are applied, for example, such that as described above, according to the spatial arrangement, slices numbered in a stacking direction are distributed into slice groups, specifically such that the slice numbers of the slices in slice groups differ by the total number of the slices divided by the acceleration factor. As a result, a list of slice groups is obtained which are sorted in an ascending or descending manner according to the lowest slice number. These slice groups are now allocated to concatenations and sequence portions of the repetition sequences in the relevant concatenations. For this purpose, in the case considered here, a plurality of concatenations are allocated to slice groups according to the list of successive different concatenations in a defined concatenation sequence. If, for example, the concatenation sequence is K1, K2, K3, the first slice group is allocated in accordance with the list to K1, the second to K2, the third to K3, the fourth to K1, the fifth to K2 and so on. In the example already discussed above of twenty-six slices, an acceleration factor of two and a concatenation number of two, the following repetitions take place:
K1: (S1,S14), (S3,S16), (S5,S18), (S7,S20), (S9,S22), (S11,S24), (S13,S26)
K2: (S2,S15), (S4,S17), (S6,S19), (S8,S21), (S10,S23), (S12,S25).

In step 9, a crosstalk condition is evaluated for the recording sequence that has been established. The crosstalk condition checks, put in general terms, whether at least one first slice that is recorded in the last sequence portion of one of the repetition sequences is spatially immediately adjacent to at least one second slice which is recorded in the first sequence portion of the same repetition sequence, so that on re-iteration of the repetition sequence, the first and the second slice would be recorded (and thus excited) temporally immediately one after another. In the example set out above, in the repetition sequence for K1, the first slice would be the slice S13 and the second slice would be the slice S14.

In the exemplary embodiment set out here, the crosstalk condition does not analyze the repetition sequences per se, but checks whether a simple logical mathematical relation which depends upon the total number of slices, the acceleration factor and the concatenation number is true. Making use of the ordering scheme, as described in relation to step 8, the mathematical relation may comprise that a first integer number which is defined as a reduced number of slices modulo a second integer number which is defined as the concatenation number is equal to one. Therein, the reduced number of slices is defined as the total number of the slices divided by the acceleration factor.

If it is ascertained in step 9 that the crosstalk condition is fulfilled and therefore that the mathematical relation is “true”, the recording sequence in step 10 is adapted according to an adaptation rule, otherwise the imaging begins in step 11 according to the originally established recording sequence of step 8.

The adaptation rule in step 10 includes that, in order to create a greatest possible temporal recording separation between the first slice and the second slice, either the slice group with the first slice or the slice group with the second slice is displaced into the center of the repetition sequence, that is, the central sequence portion, wherein however the sequence of all the other slice groups remains unchanged. (In the event of an even number of sequence portions, for the relevant concatenation, of the two central sequence portions, that which is further removed from the respective other affected slice is selected as the central sequence portion.)

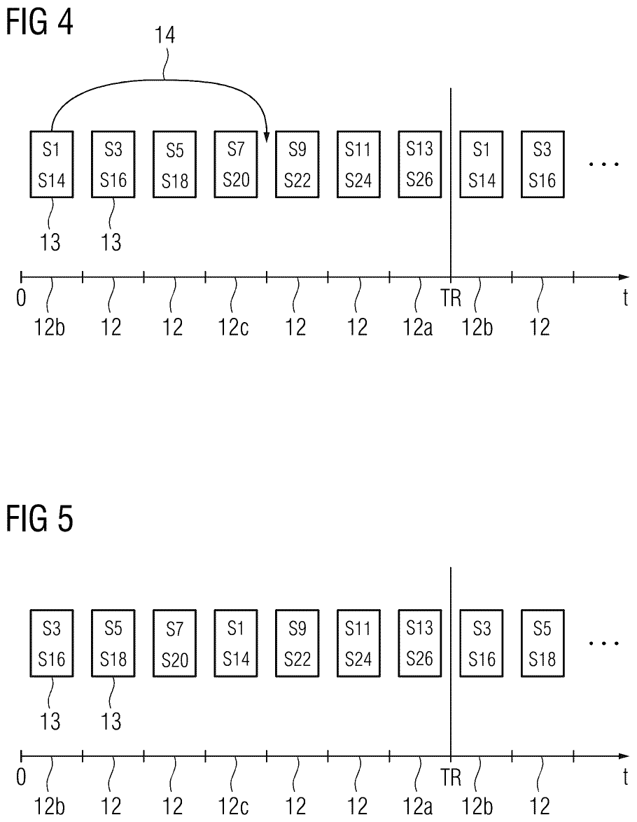

This is illustrated, by way of example, in FIGS. 4 and 5 for the repetition sequence for the concatenation K1, as set out above. Therein, it should also be noted at this point that the concatenation K1 may comprise the most slice groups and it was ascertained that at least in the determination of the recording sequence according to step 8, crosstalk problems occur, in principle, in the concatenation with the most slice groups. This concatenation then often has an odd number of slice groups, so that its central sequence portion is also clearly defined.

FIG. 4 shows that the repetition sequence given according to the recording sequence determined in step 8 is subdivided for a repetition time TR into sequence portions 12 to which corresponding slice groups 13 are allocated. In the last sequence portion 12a, slices S13 and S26 are acquired, while in the first sequence portion 12b, slices S1 and S14 are recorded. On the basis of the re-iteration of the repetition sequence, the sequence portions 12a, 12b are temporally immediately adjacent, while the slices S13 and S14 are spatially immediately adjacent. The crosstalk condition is fulfilled. In order to adapt the recording sequence, as indicated by the arrow 14 in FIG. 4, the slice group 13 which is allocated to the sequence portion 12b is displaced into the center of the repetition sequence, more precisely to the exact central sequence portion 12c. FIG. 5 shows the result of this displacement, which leaves the sequence of the other slice groups 13 untouched. The slices S1 and S14 are now recorded in the sequence portion 12c so that the temporal separation between the recording of the slices S13 and S14 is maximized.

Returning to FIG. 3, in step 11, the magnetic resonance dataset is then recorded using the adapted recording sequence. Herein, in the magnetic resonance sequence, at least one preparation module is applied, as described.

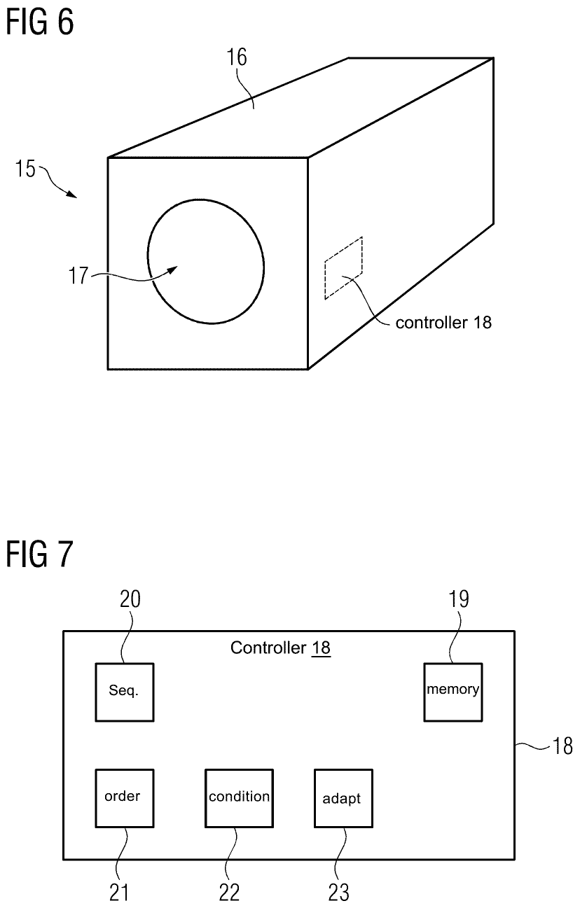

FIG. 6 shows a representation of the principle of a magnetic resonance facility (MR system) 15 according to the present disclosure. The magnetic resonance facility 15 may comprise, as is known in principle, a main magnet unit 16 with a cylindrical patient receptacle 17 for receiving a patient on a patient table (not shown). A high frequency coil arrangement and a gradient coil arrangement can be provided surrounding the patient receptacle 17. The operation of the magnetic resonance facility 15 may controlled by a control facility (controller) 18.

FIG. 7 shows the functional structure of the control facility (controller) 18. The control facility 18 may comprise a storage means (memory) 19 in which, for example, the at least one ordering rule, the adaptation rule and the crosstalk condition can also be stored as well as other data relating to the method as described and/or for the operation of the magnetic resonance facility 15. In an exemplary embodiment, the control facility (controller) 18 may include processing circuitry that is configured to perform one or more functions/operations of the controller 18. Additionally, or alternatively, one or more components of the controller 18 may include processing circuitry that is configured to perform one or respective functions/operations of the component(s).

The control facility 18 may further comprise a sequence unit 20 for control of the recording of magnetic resonance data, in particular, also in accordance with step 11.

In an ordering unit 21, the recording sequence according to step 8 can be established. In a condition unit 22, the crosstalk condition according to step 9 can be checked and, in an adapting unit 23, the adaptation on fulfillment of the crosstalk condition according to step 10 takes place.

Although the disclosure has been illustrated and described in detail with the exemplary embodiment, the disclosure is not restricted by the examples disclosed and other variations can be derived therefrom by a person skilled in the art without departing from the protective scope of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for operating a magnetic resonance facility for recording a magnetic resonance (MR) dataset including a total number of slices and using a simultaneous multislice (SMS) imaging technique, the method comprising: establishing, using at least one ordering rule, a recording sequence comprising an allocation of slices to corresponding sequence portions of a magnetic resonance sequence of at least one repetition sequence covering all the slices of at least one allocated concatenation;
evaluating a crosstalk condition for the established recording sequence, wherein the crosstalk condition checks whether at least one first slice that is recorded in a last sequence portion of at least one of the at least one repetition sequence is spatially adjacent to at least one second slice recorded in a first sequence portion of a same repetition sequence; and
in response to a fulfillment of the crosstalk condition, adapting the recording sequence to increase a temporal separation between the recording of the at least one first slice and the at least one second slice, wherein the adapting of the recording sequence comprises displacing a slice group comprising the at least one first slice or a slice group comprising the at least one second slice to a central sequence portion of the at least one repetition sequence.

2. The method as claimed in claim 1, wherein, in the magnetic resonance sequence, a preparation module with at least one preparation pulse is used as an excitation pulse and/or the at least one second slice has a high fat content.

3. The method as claimed in claim 2, wherein the preparation module is: a fat saturation module; an inversion recovery module; and/or a SPectral Attenuated Inversion Recovery (SPAIR) module.

4. The method as claimed in claim 1, wherein:
the slices are numbered according to their spatial arrangement in at least one stacking direction and a concatenation number of concatenations is used;
the at least one ordering rule defines a simultaneous recording of a plurality of the slices in slice groups such that slice numbers of the slices in each slice group differ by the total number of slices divided by an acceleration factor so that a list of the slice groups results that is sorted in ascending or descending manner according to their lowest slice number; and
to distribute the slice groups to concatenations and sequence portions:
two sublists are created one after another in response to the concatenation number being one, wherein a first of the two sublists contains all the even slice numbers and a second of the two sublists contains all the odd slice numbers, and
the slice groups are allocated according to the list, one after another, to different concatenations in a particular concatenation sequence in response to the concatenation number being greater than one.

5. The method as claimed in claim 4, wherein a reduced number of the slices is defined as the total number of slices divided by the acceleration factor and, in order to evaluate the crosstalk condition, further comprising checking whether a first integer number defined as the reduced number of slices modulo a second integer number, defined as the concatenation number, is equal to one.

6. The method as claimed in claim 1, wherein, in a sequence portion of the magnetic resonance sequence that is used, magnetic resonance signals are simultaneously acquired from a simultaneity number, that is equal to an acceleration factor and is at least two, of the slices of the slice group.

7. A magnetic resonance system comprising: a controller configured to perform the method as claimed in claim 1.

8. An apparatus comprising:
one or more processors; and
memory storing instructions that, when executed by the one or more processors, cause the apparatus to perform the method as claimed in claim 1.

9. One or more non-transitory media storing instructions that, when executed by one or more processors, cause the one or more processors to perform the method as claimed in claim 1.
